(19) United States
(12) Patent Application Publication
(10) Pub. No.: US 2010/0249103 A1
(43) Pub. Date: Sep. 30, 2010

(54) COMBINATION TREATMENT

(76) Inventors: Jukka Sallinen, Turku (FI); Mikko Kuoppamäki, Raisio (FI); Jouko Levijoki, Helsinki (FI)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/674,689

(22) PCT Filed: Aug. 29, 2008
(86) PCT No.: PCT/FI2008/000097

§ 371 Date: Apr. 6, 2010

Related U.S. Application Data
(60) Provisional application No. 60/968,583, filed on Aug. 29, 2007.

(51) Int. Cl. A61K 31/50; A61K 31/55; A61P 9/00

(52) U.S. Cl. 514/212.07; 514/247

(57) ABSTRACT

A combination of a levosimendan compound or a pharmaceutically acceptable salt thereof and an angiotensin II receptor antagonist or an angiotensin converting enzyme (ACE) inhibitor shows synergistic effect in the prevention of stroke. Pharmaceutical compositions and medical kits comprising as a first active ingredient a levosimendan compound or a pharmaceutically acceptable salt thereof and as a second active ingredient an angiotensin II receptor antagonist or an angiotensin converting enzyme (ACE) inhibitor are provided.

### TECHNICAL FIELD

The present invention relates to a method of treatment of patients using a levosimendan compound or a pharmaceutically acceptable salt thereof in combination with an angiotensin II receptor antagonist or an angiotensin converting enzyme (ACE) inhibitor. The invention also relates to a pharmaceutical compositions and medical kits comprising as a first active ingredient a levosimendan compound or a pharmaceutically acceptable salt thereof and as a second active ingredient an angiotensin II receptor antagonist or an angiotensin converting enzyme (ACE) inhibitor.

### BACKGROUND OF THE INVENTION

Stroke is the third leading cause of death and the main cause of permanent health damage in adults. High blood pressure is known to be one of the most important risk factors for acute stroke. Consequently, the risk of hypertensive patients suffering an acute stroke can be reduced through antihypertensive therapy.

Angiotensin II receptor antagonists are antihypertensive compounds that selectively block the AT₁ subtype of the angiotensin II receptor. Angiotensin II is a potent natural vasoconstrictor having blood pressure increasing effects as well as growth promoting effects contributing to left ventricular hypertrophy, vascular thickening, atherosclerosis and stroke. Angiotensin II receptor antagonists are mainly used in the treatment of high blood pressure, particularly in patients who are intolerant to ACE inhibitor therapy. Subsequently, clinical trials have indicated beneficial effects of angiotensin II receptor antagonists in the prevention of hypertensive complications such as stroke, whereby the stroke prevention effect at least partly appeared to be independent of blood pressure lowering effect.

Angiotensin II is formed from angiotensin I in the blood by an enzyme, angiotensin converting enzyme (ACE). ACE inhibitors are agents that inhibit the activity of the enzyme thereby decreasing the production of angiotensin II. ACE inhibitors are used primarily in the treatment of hypertension and congestive heart failure.

Levosimendan, which is the (−)-enantiomer of [[4-(1,4,5,6-tetrahydro-4-methyl-6-oxo-3-pyridazinyl)phenyl]hydrazono]propanedinitrile, is currently used for the short term treatment of patients who suffer from acutely decompensated severe heart failure. The drug increases contractile force of the heart myocardium by enhancing the sensitivity of myofilaments to calcium. Administration of levosimendan together with a thrombolytic agent in the treatment of acute myocardial infarction has been described in WO 99/66932. Use of levosimendan in the prevention of thrombotic, embolic and/or hemorrhagic disorders such as stroke has been described in WO 2005/107757.

Levosimendan has an active metabolite (R)—N-[4-(1,4,5,6-tetrahydro-4-methyl-6-oxo-3-pyridazinyl)phenyl]acetamide (II) which is present in man following administration of levosimendan. Its effects have been reported to be similar to levosimendan.

In spite of advances in the blood pressure control there is still need for improved therapies for angiotensin II related cardiovascular diseases such as prevention and treatment of stroke in high risk patients.

### SUMMARY OF THE INVENTION

It has now been found that administration of a levosimendan compound or a pharmaceutically acceptable salt thereof together with an angiotensin II receptor antagonist or an angiotensin converting enzyme (ACE) inhibitor provides unexpectedly synergistic effect in reducing hypertensive complications, particularly the incidence and volume of brain lesions, morbidity and mortality associated with stroke in hypertensive salt sensitive rat model. Therefore, the combination is useful in the prevention and treatment of angiotensin II related cardiovascular diseases, such as hypertensive complications. In particular, the combination is useful in the prevention or inhibition of stroke or in reducing a risk of stroke, especially in the treatment of patients at high risk of stroke, such as hypertensive patients or patients who have suffered earlier stroke.

Thus, in one aspect present invention provides pharmaceutical composition comprising as a first active ingredient a levosimendan compound or a pharmaceutically acceptable salt thereof and as a second active ingredient an angiotensin II receptor antagonist or an angiotensin converting enzyme (ACE) inhibitor.

In another aspect the present invention provides a medical kit comprising in separate containers in a single package pharmaceutical compositions comprising in one container a pharmaceutical composition comprising a levosimendan compound or a pharmaceutically acceptable salt thereof and in a second container a pharmaceutical composition comprising an angiotensin II receptor antagonist or an angiotensin converting enzyme (ACE) inhibitor.

In another aspect the present invention provides a method for prevention or inhibition of stroke or reducing a risk of stroke in a patient, which comprises the simultaneous, separate or sequential administration of an effective amount of a levosimendan compound or a pharmaceutically acceptable salt thereof and an angiotensin II receptor antagonist or an angiotensin converting enzyme (ACE) inhibitor to a patient in need thereof.

In another aspect, the present invention provides a method for prevention or inhibition of stroke or reducing a risk of stroke in a patient comprising administering to said patient an effective amount of a levosimendan compound or a pharmaceutically acceptable salt thereof in conjunction with an angiotensin II receptor antagonist or an angiotensin converting enzyme (ACE) inhibitor.

### BRIEF DESCRIPTION OF THE DRAWINGS

FIG. 1 shows the survival (%) of Dahl salt-sensitive rats on salt rich diet treated with levosimendan, valsartan or combination of levosimendan and valsartan in comparison to the control group.

FIG. 2 shows the increase of body weight of Dahl salt-sensitive rats on salt rich diet treated with levosimendan, valsartan or combination of levosimendan and valsartan in comparison to the control group, wherein the arrow denotes the start of the drug treatments.

FIG. 3 shows the gross histology score of brain pathology in Dahl salt-sensitive rats on salt rich diet treated with levosimendan, valsartan or combination of levosimendan and valsartan in comparison to the control group. Percent of animals are given as pie presentation according to the severity of brain lesions in each treatment group.

FIG. 4 shows the survival (%) of Dahl salt-sensitive rats on salt rich diet treated with levosimendan, losartan or combination of levosimendan and losartan in comparison to the control group.

FIG. 5 shows the survival (%) of Dahl salt-sensitive rats on salt rich diet treated with ramipril or combination of levosimendan and ramipril in comparison to the control group.

### DETAILED DESCRIPTION OF THE INVENTION

The method of the invention relates to a combination therapy for more effective treatment of angiotensin II related cardiovascular diseases. In particular, the present invention relates to a combination therapy for more effective treatment in the prevention or inhibition of stroke. According to one preferred embodiment of the invention, the combination treatment of the invention is able to synergistically reduce the incidence or the recurrence of stroke, reduce the severity of stroke or reduce the mortality and/or morbidity associated with stroke.

As used herein the term “stroke” means a cerebrovascular accident (CVA), particularly the sudden death of some brain cells due to lack of oxygen when the blood flow to the brain is impaired by blockage or rupture of an artery to the brain. Examples of stroke include cerebral thrombosis, cerebral embolism, intracerebral hemorrhage (ICH), subarachnoid hemorrhage (SAH), transient ischemic attack (TIA) and vascular dementia.

The term “prevention of stroke” means reducing the incidence or the recurrence of stroke. The term “patient” means animals, preferably mammals, and humans.

According to one embodiment of the invention, a levosimendan compound or a pharmaceutically acceptable salt thereof is administered together with an angiotensin II receptor antagonist or an angiotensin converting enzyme (ACE) inhibitor for providing prevention or inhibition of stroke or reduction a risk of stroke.

The method of invention is particularly useful for the treatment of individuals having high risk of stroke. Conditions which are associated with high risk of stroke include, but are not limited to, earlier stroke; hypertension; diabetes; earlier heart attack; heart disease; orthopaedic fractures or other injuries; prolonged bed rest; elevated blood lipid levels; atherosclerosis; and peri- and postoperative periods of surgical operations.

According one embodiment of the invention, a levosimendan compound or a pharmaceutically acceptable salt thereof is used in conjunction with an angiotensin II receptor antagonist or an angiotensin converting enzyme (ACE) inhibitor for the prevention or inhibition of stroke or in reducing a risk of stroke independent of lowering elevated blood pressure. Patients to be treated may or, according to another embodiment of the invention, may not suffer from hypertension.

The active ingredients may be administered simultaneously, separately or sequentially. In particular, the method comprises administering to a patient an amount of active ingredients or combination thereof which is effective for prevention or inhibition of stroke or reducing a risk of stroke in the patient. Preferably, the method comprises administering to a patient a synergistically effective amount of the combination. The administration routes of the active ingredients include, but are not limited to, enteral, e.g. oral or rectal, or parenteral, e.g. intravenous, intramuscular, intraperitoneal or transdermal. For the prevention of stroke, oral administration of the active ingredients is particularly preferred.

As used herein, the term “levosimendan compound” refers to any racemic mixture or enantiomer of levosimendan or a racemic mixture or enantiomer of the active metabolite of levosimendan. The term “levosimendan” specifically refers to the (−)-enantiomer of [4-(1,4,5,6-tetrahydro-4-methyl-6-oxo-3-pyridazinyl)phenyl]-hydrazono]propanedinitrile. The term also is intended to encompass combinations of levosimendan and its active metabolite. The active metabolite of levosimendan is particularly (R)—N-[4-(1,4,5,6-tetrahydro-4-methyl-6-oxo-3-pyridazinyl)phenyl]-acetamide (II). Particularly preferred active ingredient is levosimendan or a pharmaceutically acceptable salt thereof.

Levosimendan or its active metabolite may suitably be administered orally to man in a daily dosage ranging from about 0.05 to 10 mg, preferably from about 0.1 to 5 mg, depending on the route of administration, age, weight and the condition of the patient given once a day or divided into several doses a day. For the long-term prevention of stroke in man, relatively low oral doses are generally preferred, e.g. an oral daily dose from about 0.05 to about 5 mg, preferably from about 0.1 to about 4 mg, more preferably from about 0.2 to about 3 mg.

Levosimendan can be administered by intravenous infusion using the infusion rate from about 0.01 to 5 μg/kg/min, typically from about 0.02 to 3 μg/kg/min, for example from about 0.05 to 0.4 μg/kg/min. The active metabolite can be administered intravenously using an infusion rate, which is from about 0.001 to about 1 μg/kg/min, preferably from about 0.005 to about 0.5 μg/kg/min.

Any angiotensin II receptor antagonist or an angiotensin converting enzyme (ACE) inhibitor known in the art may be used in the method of the invention in combination with a levosimendan compound. Examples of suitable angiotensin II receptor antagonists include sartans such as losartan, valsartan, telmisartan, candesartan, eprosartan, irbesartan, olmesartan, tasosartan and pharmaceutically acceptable salts thereof. Examples of suitable angiotensin converting enzyme (ACE) inhibitors include ramipril, captopril, enalapril, quinapril, perindopril, lisinopril, benazepril, moexipril, trandolapril and pharmaceutically acceptable salts thereof.

According to the invention, angiotensin II receptor antagonists may be administered in daily doses which are clinically accepted for such agents. For example, the angiotensin II receptor antagonist may suitably be administered orally to man in a daily dosage ranging from about 2 to about 600 mg, for example from 20 mg to 300 mg, depending upon the condition to be treated, the route of administration, age, weight and the condition of the patient, and the angiotensin II receptor antagonists used.

According to the invention, angiotensin converting enzyme (ACE) inhibitor may be administered in daily doses which are clinically accepted for such agents. For example, the angiotensin converting enzyme (ACE) inhibitor may suitably be administered orally to man in a daily dosage ranging from about 1 to about 150 mg, for example from 2 mg to 80 mg, depending upon the condition to be treated, the route of administration, age, weight and the condition of the patient, and the (ACE) inhibitor used.

A specific method of prevention according to the present invention comprises administering orally 0.05-5 mg of a levosimendan compound or a pharmaceutically acceptable salt thereof and 2-600 mg of an angiotensin II receptor antagonist daily to a patient, for example 0.2-3 mg of levosimendan and 40-320 mg of valsartan, or 0.2-3 mg of levosimendan and 25-100 mg of losartan, in oral daily dosage.

A further specific method of prevention according to the present invention comprises administering orally 0.05-5 mg of a levosimendan compound or a pharmaceutically acceptable salt thereof and 1-150 mg of an angiotensin converting enzyme (ACE) inhibitor daily to a patient, for example 0.2-3 mg of levosimendan and 1-10 mg of ramipril in oral daily dosage.

The combination may be supplemented with one or more other active ingredients.

The active ingredients can be formulated into pharmaceutical dosage forms suitable for the treatment according to the present invention using the principles known in the art. They are given to a patient as such or preferably in combination with suitable pharmaceutical excipients in the form of tablets, granules, capsules, suppositories, emulsions, suspensions or solutions whereby the contents of the active compound in the formulation is from about 0.5 to 100% per weight. Choosing suitable ingredients for the composition is a routine for those of ordinary skill in the art. It is evident that suitable carriers, solvents, gel forming ingredients, dispersion forming ingredients, antioxidants, colours, sweeteners, wetting compounds, release controlling components and other ingredients normally used in this field of technology may be also used.

The active ingredients may be formulated in the same pharmaceutical formulation. Alternatively, the active ingredients are formulated as separate pharmaceutical dosage forms. The combination of the pharmaceutical dosage forms may be packaged as a single medical product or kit for use in the method of the invention, optionally together with a package insert instructing to the correct use of the medical product.

For example, according to one embodiment of the invention, the invention provides a medical product in the form of a kit comprising a first pharmaceutical dosage form comprising a levosimendan compound or a pharmaceutically acceptable salt thereof, a second pharmaceutical dosage form comprising an angiotensin II receptor antagonist or an angiotensin converting enzyme (ACE) inhibitor, a package for containing said first and second dosage forms, and optionally instructions for simultaneous, separate or sequential administration of said first and second dosage forms to a patient.

Formulations suitable for intravenous administration such as injection or infusion formulation, comprise sterile isotonic solutions of the active ingredient or active ingredients and vehicle, preferably aqueous solutions. Typically an intravenous infusion solution of a levosimendan compound comprises from about 0.01 to 0.1 mg/ml of a levosimendan compound. The pharmaceutical formulation may be also in the form of an intravenous infusion concentrate to be diluted with an aqueous vehicle before use. Such concentrate may comprise as a vehicle a pharmaceutically acceptable organic solvent such as dehydrated ethanol.

For oral administration of the active ingredients in tablet form, suitable carriers and excipients include e.g. lactose, corn starch, magnesium stearate, calcium phosphate and talc. For oral administration in capsule form, useful carriers and excipients include e.g. lactose, corn starch, magnesium stearate and talc. For controlled release oral compositions release controlling components can be used. Typical release controlling components include hydrophilic gel forming polymers such as hydroxypropylmethyl cellulose, hydroxypropyl cellulose, carboxymethyl celluloses, alginic acid or a mixture thereof; vegetable fats and oils including vegetable solid oils such as hydrogenated soybean oil, hardened castor oil or castor seed oil (sold under trade name Cutina BR), cotton seed oil (sold under the trade names Sterotex or Lubritab) or a mixture thereof; fatty acid esters such as triglycerides of saturated fatty acids or their mixtures e.g. glyceryl tristearates, glyceryl tripalmitates, glyceryl trimyristates, glyceryl tribehenates (sold under the trade name Compritol) and glyceryl palmitostearic acid ester.

Tablets can be prepared by mixing the active ingredient or active ingredients with the carriers and excipients and compressing the powdery mixture into tablets. Capsules can be prepared by mixing the active ingredient with the carriers and excipients and placing the powdery mixture in capsules, e.g. hard gelatin capsules. Typically a tablet or a capsule comprises from about 0.1 to 10 mg, more typically 0.2 to 5 mg, of a levosimendan compound or/and from about 40 to 320 mg of valsartan, from about 25 to 100 mg of losartan, from about 20 to 80 mg of telmisartan, from about 2 to 32 mg of candesartan or from about 300 to 600 mg of eprosartan, or from about 10 to 40 mg of olmesartan.

Alternatively, a tablet or a capsule comprises typically from about 0.1 to 10 mg, more typically 0.2 to 5 mg, of a levosimendan compound or/and from about 25 to 100 mg of ramipril, from about 20 to 80 mg of benazepril, from about 2 to 20 mg of enalapril maleate, from about 12 to 100 mg of captopril, from about 10 to 25 mg of quinalapril hydrochloride, from about 5 to 80 mg of lisinopril, or from about 4 to 8 mg of perindopril.

The angiotensin II receptor antagonist or the angiotensin converting enzyme (ACE) inhibitor may be included in the levosimendan formulation or may be formulated separately as described above using principles well known in the art. For example, a composition according the present invention, e.g. a tablet or capsule, may comprise from 0.2 to 3 mg of levosimendan and from 40 to 320 mg of valsartan, or from 0.2 to 3 mg of levosimendan and from 1 to 10 mg of ramipril.

Salts of levosimendan may be prepared by known methods. Pharmaceutically acceptable salts are useful as active medicaments, however, preferred salts are the salts with alkali or alkaline earth metals.

### EXAMPLES

### Pharmaceutical Examples

### Example 1

### Concentrate Solution for Intravenous Infusion

| | | |
|---|---|---|
| (a) levosimendan | 2.5 | mg/ml |
| (b) Kollidon PF12 | 10 | mg/ml |
| (c) citric acid | 2 | mg/ml |

| | |
|---|---|
| (d) dehydrated ethanol | ad 1 ml (785 mg) |

The concentrate solution was prepared by dissolving citric acid, Kollidon PF121 and levosimendan to dehydrated ethanol in the sterilized preparation vessel under stirring. The resulting bulk solution was filtered through a sterile filter (0.22 μm). The sterile filtered bulk solution was then aseptically filled into 8 ml and 10 ml injection vials (with 5 ml and 10 ml filling volumes) and closed with rubber closures.

The concentrate solution for intravenous infusion is diluted with an aqueous vehicle before use. Typically the concentrate solution is diluted with aqueous isotonic vehicles, such as 5% glucose solution or 0.9% NaCl solution so as to obtain an aqueous intravenous solution, wherein the amount of levosimendan is generally within the range of about 0.001-1.0 mg/ml, preferably about 0.01-0.1 mg/ml.

### Example 2

Hard gelatin capsule size 3

| | | |
|---|---|---|
| Levosimendan | 2.0 | mg |
| Lactose | 198 | mg |

The pharmaceutical preparation in the form of a capsule was prepared by mixing levosimendan with lactose and placing the powdery mixture in hard gelatin capsule.

### Example 3

Hard gelatin capsule size 3

| | | |
|---|---|---|
| Valsartan | 40.0 | mg |
| Levosimendan | 1.0 | mg |
| Lactose | 159 | mg |

### Example 4

Hard gelatin capsule size 3

| | | |
|---|---|---|
| Ramipril | 10.0 | mg |
| Levosimendan | 1.0 | mg |
| Lactose | 189 | mg |

Experiments

Experiment 1.

Effects of Levosimendan and an Angiotensin II Receptor Antagonist Alone and in Combination in Salt Sensitive Rat Model

Effect of levosimendan and valsartan on survival, weight gain and stroke incidence in Dahl salt-sensitive rats (Dahl SS) was studied. Dahl salt-sensitive rats on high salt diet develop hypertension and increased mortality. In the early stages of hypertension the incidence of death is almost entirely due to stroke and sudden death. See Qu, P. et al., Hypertens. Res., 2000; 23:613-623.

Four groups of 5-6-week-old male SS/JrHsd Dahl salt-sensitive rats (180 total+6 sentinel rats) received salt rich diet until 13% mortality was observed in the studied population of rats (approximately four weeks). Thereafter the rats received the following drug regimens up to 36 days while continuing on salt rich diet:

1) normal drinking water (n=20),
2) valsartan 10 mg/kg/day in drinking water (n=46),
3) levosimendan 1 mg/kg/day in drinking water (n=45), and
4) valsartan 10 mg/kg/day and levosimendan 1 mg/kg/day in drinking water (n=46).

a) Survival

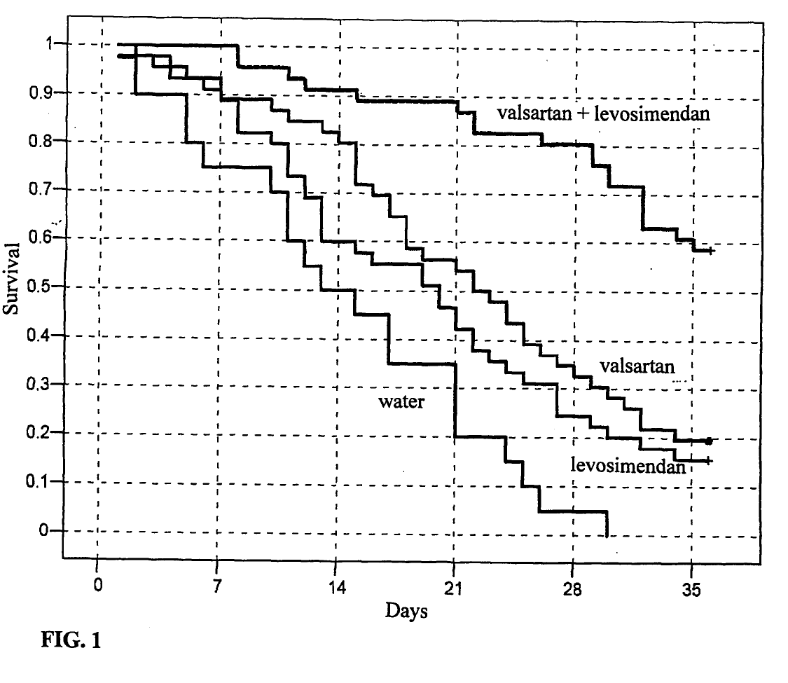

The survival results are shown in FIG. 1 as Kaplan-Meier Plot. Levosimendan and valsartan as a combination lengthened the survival highly significantly when compared to all other groups. Levosimendan and valsartan appeared to have a beneficial synergistic effect on stroke-related mortality.

b) Weight Gain

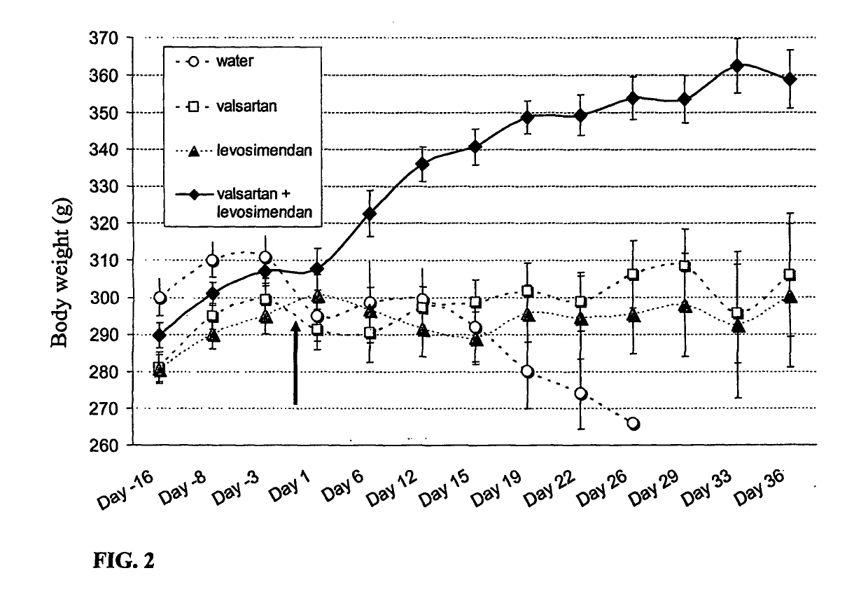

The mean body weight of the rats in each group during the study is shown in FIG. 2. The arrow denotes the start of the drug treatments. The increase in body weight that is normal in laboratory rats was evident during the salt diet period prior to drug treatments. However, the weight gain was strongly reduced at the time when mortality began to occur (about one week prior to the start of drug treatments). Only those rats that received the combined levosimendan and valsartan continued to gain further weight approaching to the weight of the sentinel Dahl SS rats in the study room that were not receiving salt rich diet.

c) Brain Pathology

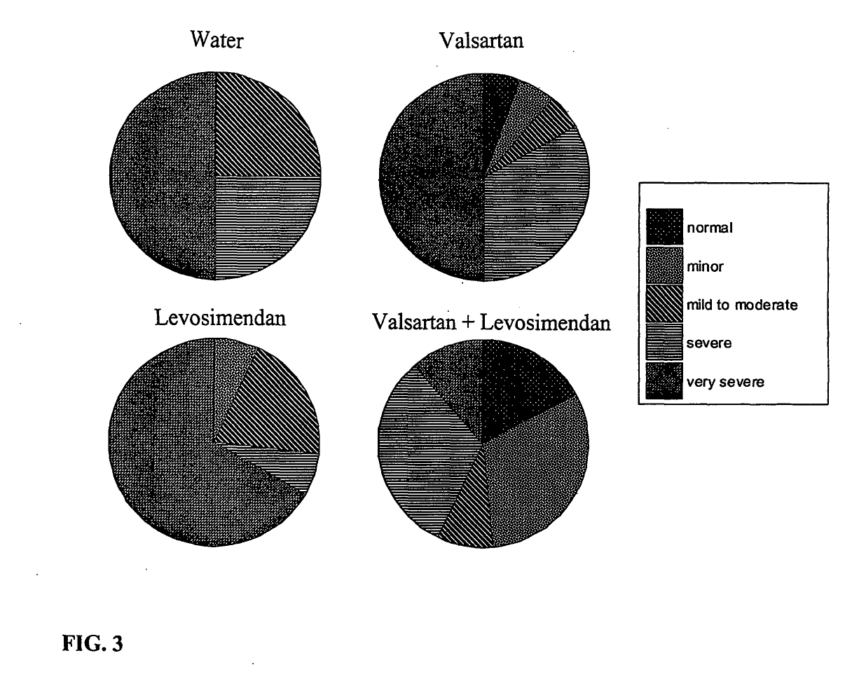

Histopathological analyses of the brain were performed for all rats that survived at least three weeks from the beginning of the drug treatments. Brains were sectioned and stained using standard methods and were subjected to microscopic analyses. The observed changes were scored (normal, minor lesions, mild to moderate lesions, severe lesions or very severe lesions). The results for each treatment group are presented in FIG. 3. It can be seen that the combination of levosimendan and valsartan reduced the incidence and volume of brain lesions associated with cerebral strokes in much higher extent than the levosimendan or valsartan alone.

Experiment 2.

Effects of Levosimendan and Losartan Alone and in Combination in Salt Sensitive Rat Model

Four groups of 5-6-week-old male SS/JrHsd Dahl salt-sensitive rats received salt rich diet until 13% mortality was observed in the studied population of rats (approximately four weeks). Thereafter the rats received the following drug regimens up to 77 days while continuing on salt rich diet:

1) normal drinking water,
2) losartan 30 mg/kg/day in drinking water,
3) levosimendan 1 mg/kg/day in drinking water, and
4) losartan 30 mg/kg/day and levosimendan 1 mg/kg/day in drinking water.

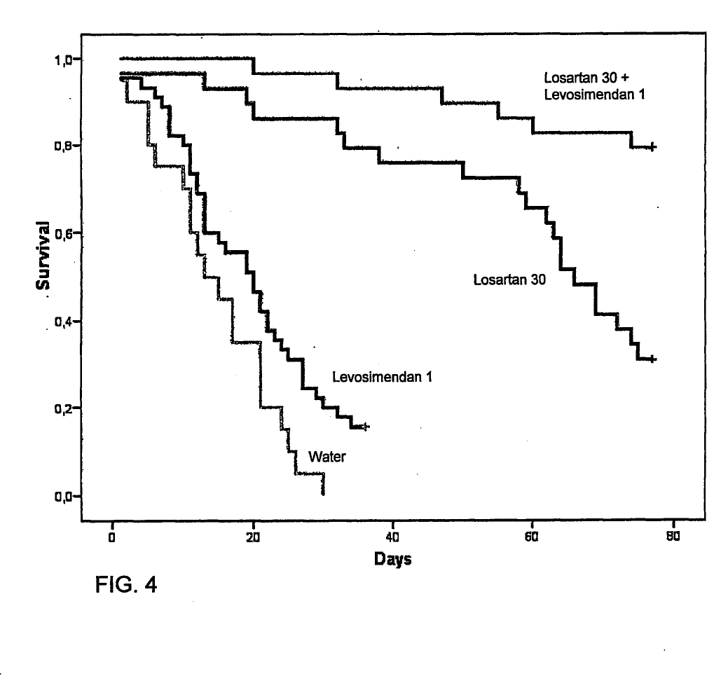

The survival results are shown in FIG. 4 as Kaplan-Meier Plot.

Levosimendan and losartan appeared to have a beneficial synergistic effect on stroke-related mortality.

Experiment 3.

Effects of Levosimendan and Ramipril Alone and in Combination in Salt Sensitive Rat Model

Three groups of 5-6-week-old male SS/JrHsd Dahl salt-sensitive rats received salt rich diet until 13% mortality was observed in the studied population of rats (approximately four weeks). Thereafter the rats received the following drug regimens up to 70 days while continuing on salt rich diet:

1) normal drinking water (n=10),
2) ramipril 1 mg/kg/day in drinking water (n=23),
3) ramipril 1 mg/kg/day and levosimendan 1 mg/kg/day in drinking water (n=22).

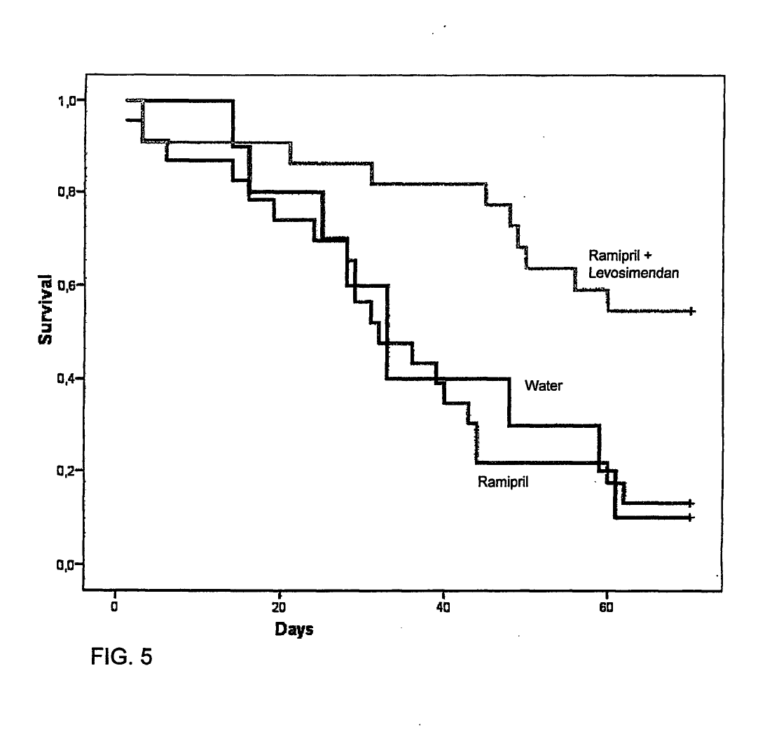

The survival results are shown in FIG. 5 as Kaplan-Meier Plot. Levosimendan and ramipril appeared to have a beneficial synergistic effect on stroke-related mortality.

1. A pharmaceutical composition comprising as a first active ingredient a levosimendan compound or a pharmaceutically acceptable salt thereof and as a second active ingredient an angiotensin IT receptor antagonist or an angiotensin converting enzyme (ACE) inhibitor.

2. The composition according to claim 1, wherein the second active ingredient is an angiotensin IT receptor antagonist.

3. The composition according to claim 1, wherein the second active ingredient is an angiotensin converting enzyme (ACE) inhibitor.

4. The composition according to claim 2, wherein the angiotensin IT receptor antagonist is losartan, valsartan, telmisartan, candesartan, eprosartan, irbesartan, olmesartan, tasosartan or a pharmaceutically acceptable salt thereof.

5. The composition according to claim 3, wherein the angiotensin convertingenzyme (ACE) inhibitor is ramipril, captopril, enalapril, quinapril, perindopril, lisinopril, benazepril, moexipril, trandolapril or a pharmaceutically acceptable salt thereof.

6. A medical kit comprising in separate containers in a single package pharmaceutical compositions comprising in one container a pharmaceutical composition comprising a levosimendan compound or a pharmaceutically acceptable salt thereof, and in a second container a pharmaceutical composition comprising an angiotensin IT receptor antagonist or an angiotensin converting enzyme (ACE) inhibitor.

7. The kit according to claim 6, wherein the angiotensin IT receptor antagonist is losartan, valsartan, telmisartan, candesartan, eprosartan, irbesartan, olmesartan, tasosartan or a pharmaceutically acceptable salt thereof.

8. The kit according to claim 6, wherein the angiotensin converting enzyme (ACE) inhibitor is ramipril, captopril, enalapril, quinapril, perindopril, lisinopril, benazepril, moexipril, trandolapril or a pharmaceutically acceptable salt thereof.

9. A method for prevention or inhibition of stroke or reducing a risk of stroke in a patient comprising administering to said patient a levosimendan compound or a pharmaceutically acceptable salt thereof in conjunction with angiotensin IT receptor antagonist or an angiotensin converting enzyme (ACE) inhibitor.

10-11. (canceled)
